(19) United States
(12) Patent Application Publication
(10) Pub. No.: US 2007/0224169 A1
(43) Pub. Date: Sep. 27, 2007

(54) Selectively switched gels for surgery, therapy and maintenance

(76) Inventors: John W. Sliwa JR., Los Altos, CA (US); Carol A. Tosaya, Los Altos, CA (US); Bryan Oronsky, Los Altos, CA (US)

Correspondence Address: David W. Collins;Intellectual Property Law, Suite 100, 512 E. Whitehouse Canyon Road, Green Valley, AZ, 85614, US

(21) Appl. No.: 11/488,945

(22) Filed: Jul. 18, 2006

Related U.S. Application Data
(60) Provisional application No. 60/786,780, filed on Mar. 27, 2006.

(51) Int. Cl. A61K 48/00; A61K 9/00

(52) U.S. Cl. 424/93.2; 424/400

(57) ABSTRACT

A gel-like material is provided, the gel-like material having an on-demand state-change capability, whether the on-demand change is to an initial state, a final state or both, at least some of the gel present or delivered undergoing the change. Alternatively, the gel-like material may have a spatially and/or temporally selective state-change, whether on-demand change is to an initial state, a final state or both, at least some of the gel present or delivered undergoing the change. Or, the gel-like material may selectively change state due to gel contacting diseased tissue having a natural thermal or compositional contrast or artificially induced thermal or compositional contrast capable of causing state-change via a state-change parameter, independent of an on-demand nature or a spatial/temporal selective nature.

### CROSS-REFERENCE TO RELATED APPLICATION

The present application claims priority from provisional application Ser. No. 60/786,780, filed Mar. 27, 2006.

### BACKGROUND OF THE INVENTION

A. The Prior Art and History of Gels in General

Excellent reviews of historic gels, particularly natural protein and sugar-based gels used across the food and cosmetics industries by the tons for decades and even centuries, are given by the following references:

Reference #1, “Formulating by Gum, Pectin and Gelatin” by Lynn A. Kuntz, Food Product Design: Applications—June 2002 (www.foodproductdesign.com/archive/2002/0602AP.html);

Reference #2, “GEL, A Short Word With A Long Meaning” by Susana B. Grassino, (www.pslc.ws/macrog/property/gel/gel.htm);

Reference #3 “Definitions Of Terms Relating To The Structure And Processing Of Inorganic And Polymeric Gels and Networks, And Inorganic-Polymeric Materials”, by IUPAC, Provisional IUPAC Document—14^{th} Draft, 10 Feb. 2006 (Note that Ref #3 lists prior art definitions of gels and related materials and not the broader definition given below for the purpose of our inventive teaching herein; thus, all gel or gel-like materials referred to in Ref #3 constitute a subset of our broader inventive materials scope);

Reference #4 “Gels and Starch Gels”, The Technology Information Sheet Of Innogel PLC;

Reference #5 “Polymeric Gels For Improved Drug Delivery” by John Cleary (www.ul.ie/elements/Issue6/Polymeric%20gels.htm);

Reference #6 “Theramer Technology”, Rimon Therapeutics Website (www.rimontherapeutics.com/theramer_products.htm);

Reference #7 “Pressure Dependent Phase Behavior Of Polymer Gels” by Mitsuhiro Shibayama, Proceedings of the International Symposium on Research Reactor and Neutron Science, Korea, April 2005;

Reference #8 “MacroMed Technology: ReGel® Injectable Gel Depot” (www.macromed.com/regel.htm);

Reference #9 “Thermosensitive Polymer Delivery-Thermally Reversible Gelling Materials For Safe And Versatile Depot Delivery” by Kirk D. Flowers et al (www.drugdeliverytech.com/cgi-bin/articles.cgi?idArticle=154);

Reference #10 “Mebiol Bio Products: Mebiol Gel”, www.mebiol.co.ip/english/medical.htm).

Gels or gel-like materials are typically elastic, plastic, viscoelastic, viscoplastic or thixotropic deformable materials made from liquid precursors. Typically, they are at least elastic and somewhat viscoelastic for moderate deformations. For larger deformations, they are typically also viscoplastic. A few are thixotropic with modest elastic behavior. They represent a material having atomic order between that of liquids and solids. They are very frequently comprised of a matrix, cellular or micelle structures wherein the cells or micelles contain liquid components. Such structures are referred to collectively as network or networked structures by Materials Scientists. Such cells or micelles may range in size from nanometers to microns or larger. They may or may not allow for bulk permeability by particular ions, atoms, molecules or particles, typically depending on the particle size and electrical charge of the intended mobile species. The cells and/or their contents might be hydrophilic or hydrophobic, individually or taken together.

A good overview of gels is given by the above Reference #2 “Gels-A short word with a long meaning”, by Susana Grassino. Gels may be organic-based or inorganic based as are sol-gels which are gel precursors. A second good gel overview is given by that of Inno-Gel as Reference #4 above.

Gels that can reversibly thermally (or under the influence of ions, pressure, solvents, etc.) change between liquid-like (flowable) and gel-like (pseudosolid) are referred to as thermoreversible gels. Note that they may become liquid upon heating and solid-like upon subsequent cooling in a manner analogous to freezing or crystallization. Others may become solid-like when heated but liquid-like again upon subsequent cooling in a manner opposite that of familiar freezing or crystallization. Some gels that gel from liquids upon warming include those based on polyoxyethylene and polyoxypropylene compounds. Note that by the verb “gel” herein we simply mean taking on a less-flowable or unflowable state. Per our gel-like material definition below, we include many materials not before called “gels” nor necessarily meeting prior art structural networking technical definitions of gels from the microstructure viewpoint.

Thermally-reversible or thermoreversible gels, within useful designed temperature ranges, can therefore be purposely thermally switched from liquid-like to solid-like several times or more. This switching of flowability state may happen over a narrow temperature range of a few degrees C. or over a broader temperature range of, say, 20° C., depending on gel design.

Solvent-reversible gels typically assume their unflowable state upon solvent extraction and can reassume a flowable liquid state upon reintroduction or reabsorbance of such solvent. In many cases, ionic concentration changes in a gel will trigger unflowability or flowability with or without bulk solvent transport. We also consider this to be reversible behavior. In many cases, as for hydrogels, the solvent is water. Thus, one might dissolve a solidified gel simply by doping the water already surrounding it with ions, as opposed to presenting undoped or doped water to a gel.

Some gels are permanently gelled in that temperature (or ionic, solvent, pressure, etc.) manipulation cannot cause the above reversible changes. Generally speaking, gels wherein the molecular constituents have covalent (chemical) bonds are not thermoreversible or reversible, whereas gels having intermolecular tangling or weak bonds such as van der Waals or hydrogen bonds (physical bonds) can be thermoreversible or reversibly dissolvable. Many gels are protein-water systems or polymer-solvent systems. Thermoreversible gels, when changing from pseudosolid to liquid-like, go through what is called the sol-gel transition temperature and this temperature or temperature range depends on the concentration of the protein or polymer in the water or solvent. It may instead or also depend on an ionic concentration.

Gels or gel-like materials, again frequently comprising networks of molecules surrounding solvent-filled or water-filled miscelles, may vary from hard to “barely-hangs-together”. In general, the denser the network and the less the swelling (solvent loading), the harder the gel will be and the lower the network density and the higher the swelling, the softer the gel will be. Many gels will take up solvents if they are available, thereby increasing their swelling. Hydrogels certainly take up water for example. Such materials make good absorbers, such as of chemical spill or of a drug to be delivered to tissues. An example of a soft gel would be the dessert Jell-O®, whereas an example of a “harder” gel would be rubber. Examples of in-between hardness natural gel materials include brain-matter and subsurface skin-tissue.

Many water-based systems are called hydrogels. Hydrogels, as well as many polymer-based gels, are frequently biocompatible and protein-friendly. PVA-based hydrogels are an example of such materials.

Thus, we have gels that can be solid/flowable reversible via temperature, pressure, ion or solvent exposures or combinations of these. In the future, we further expect gels which can be reversed by electrical, magnetic and optical fields or exposure thereto.

B. Prior Art Medical Gels In Particular

The gels used today for medical purposes fall into a few categories and can be generally grouped by what form they are delivered-in and/or what form they are utilized in. Some examples follow:

a) Hand-applied to the skin or mucous-membrane as a gel deposit or gel depot. The gel itself delivers a beneficial effect or a drug or medicament in the gel, diffusing outwards into tissue and providing a beneficial effect. The gel may be arranged to biodegrade over time and may also be arranged to deliver a drug over time as by diffusion or bio-breakdown. Anti-HIV gels act as HIV barriers and may also have drugs or medicaments in them. A drug-loaded gel skin patch is an external gel-depot example for controlled drug delivery. Alternatively, a sun-block gel may convert to liquid upon skin application, the gel state simply minimizing application messiness.

b) Injected or diffused as a liquid, and may or may not contain a drug or medicament. One implementation of this is a liquid-like injectable gel that undergoes unavoidable and desirable gellation (pseudo-solidification) after injection due to the higher temperature of the body than the ambient. Such a “solidifying” injection may serve to slowly deliver drugs or medicaments over time as they diffuse or leach out of the in-situ solidified gel. The solidified gel is relatively immobile as a mass.

c) Surgically implanted as a gel, as in the surgical implantation of a gel-like or semisolid drug-containing implant adjacent or inside a tumor to be killed. May biodegrade, diffuse drugs or gradually dissolve and deliver drugs. The point here is the implantation of a shaped depot which is solidified before or during surgical implantation.

d) As an in-vitro (on the bench) gel-culture substrate: Numerous biological and genetic species have been demonstrated to be able to thrive, grow, multiply and/or at least survive in gel ambients. This phenomenon is being extended by researchers to culture and growth of biological species in the body itself.

e) As a gel diffusion substrate: As for electrophoresis analysis done in a biolab In all of these cases, if there is a gel-to-liquid or liquid-to-gel thermal transition, it happens to all of the gel at one time such as when liquid (gel) is injected into a tumor. Our invention herein applies a new degree of freedom that is selective thermal (or other state-change parameter) transformations, meaning selective in spatial coordinates and/or time and/or on-demand.

An example of a gel that solidifies with warming is Pluronic-PAA from the University of Limerick (see John Cleary Reference #5). An example of a company that makes gels that, even when not loaded with drugs, provide a therapeutic effect is Rimon Therapeutics in Toronto, Canada (see Rimon website at www.rimontherapeutics.com or Reference #6).

Many gels are optically transparent or nearly so. Many others are bioadhesive, meaning they bond to adjacent tissues and/or inherently support cellular growth as do tissue scaffold structures.

Some researchers attempt to grow crystals of protein and other biological or non-biological materials in gels such as in agarose gel, as it can be correctly argued that a gel provides an ideal environment for such unperturbed growth and acts as an endless source for crystallizing species. Such gels could also support precipitation reactions, as can liquid-solute systems, thus gels can be used to “grow” or nucleate crystals or compounds. This could also be done in the human body.

Many gels exhibit significant and reversible changes in structure, volume, stiffness, etc. when an environmental parameter is changed, causing a physical parameter within the gel to change in response. Such environmental parameters include temperature, pH, solvent exposure, ionic species or ion-exposure (such as to calcium ions), light exposure, pressure exposure, and exposure to electric or magnetic fields. (Reference #7 “Pressure dependent phase behavior of polymer gels” by Shibayama gives examples of pressure dependent gels in particular.) Typically, the effect of the external environmental change is carried into or diffused into the gel.

Drug release from gels can be driven as by changing their microstructure using applied temperature changes and/or pH changes, for example. Drug release can also be caused by passive outdiffusion driven by, for example, a drug concentration gradient relative to target tissue. A further method of delivering drugs or any other species from a gel is to photo-expose the gel. Many gels exhibit breakdown and/or physical changes with such light input. A particular advantage of light is that pulsed flow of drugs becomes possible. Ultrasound is also known to both be able to liberate drugs from gels as well as to drive the diffusion thereof out of gels and into tissues.

It is vital now that we mention the use of microparticles, microspheres and nanoparticles in gels. Such particles can themselves be deliverable or active therapeutic species or may act as containers or shells enclosing drug or radiation species. If they act as containers for a deliverable or active species, then their shells may provide another release gate (e.g., for a diffusing drug) whereat the rate or suddenness of drug release can be affected. In this manner, the gel may be independently optimized for other desirable properties such as gellation and biocompatibility as opposed to drug-diffusion control.

C. Specific Prior Art for Medical Gels

The relevant prior art generally involves the utilization of gels or similar polymers in the body for purposes of delivering drugs or therapeutic radiation over an extended period of weeks. In particular, gels have recently been developed which solidify upon warming and these gels are liquid-injected using a syringe and they immediately become permanently unflowable by exposure to the 37° C. or so heat of the body upon injection into warm tissues or bodily fluids. Typically, such a gel would carry a drug and act as a drug depot for a surrounding or adjacent tumor. Such gels are typically ultimately removed from the body by natural biodegradation processes over a period of weeks or months. In some cases, gels can be designed that reliquefy upon cooling. These are currently being used only to grow cells in culture dishes. In addition to these gels that solidify when warmed and liquefy when cooled again, there are numerous historic gels that do the opposite, namely, solidify during cooling and/or solvent removal. We shall now give some specific examples of all of these.

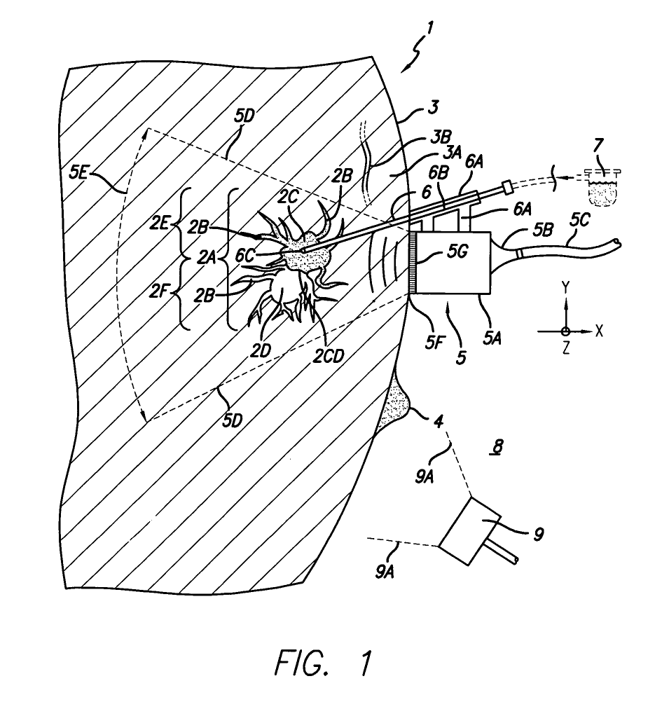

Regel™ by MacroMed® is based on a triblock copolymer composed of poly(lactide-co-glycolide) A blocks and poly(ethylene glycol) B blocks (Reference #8). Reference #9 entitled “Thermosensitive Polymer Delivery-Thermally Reversible Gelling Materials For Safe And Versatile Depot Delivery” describes its manufacturing process and properties. Of greatest importance is their FIG. 2 therein, which shows that the liquid-to-semisolid conversion takes place over a very narrow temperature range of 2° C. or so. Further therein, their FIG. 5 demonstrates that by changing the A/B ratio, one can set a desired gellation temperature or T_{gel}. The Regel™ products on the market have T_{gel} temperatures below 37° C. (body temperature) and therefore they immediately and unavoidably solidify upon injection into the body. This is a highly desirable attribute for a drug delivery depot being positioned in a tumor for a period of weeks. MacroMed also has its OncoGel™ anticancer product, which is essentially Regel™ with a cancer drug loaded into the gel. Regel™ naturally biodegrades over a period of weeks; thus, it is in the gel state in the body for weeks or even months and cannot and is not desired to be immediately removed or removed on-demand.

Macromed™ also has two drug-delivery or drug-dissolution liquid media based on similar gellation-capable chemistries called Hysolv® and Resolv®. However, it must be emphasized that these media are not currently used in the gel state. It appears that these are similar gellation-capable materials as Regel™; however, their gellation temperature are purposely chosen to be above 37° C., as they are used solely in liquid form for their huge advantage in liquid-based drug-dissolution ability. In other words, they would reversibly gel if heated above 37° C. by several degrees C but they are used instead as liquid-phase dissolving agents at 37° C. or below. It is a common trait of many gellation-capable material systems that they can dissolve large amounts of drugs even in their liquid state in which they are used.

Mebiol® gel from Mebiol Inc. in Japan is described in Reference #10 entitled “Medical Bio Products-Mebiol® Gel”. The details of its composition are not given; however, it is a hydrogel and its liquid-to-solid and solid-to-liquid changing behavior is reversible. The existing Mebiol® product has a T_{gel} in the range of 20 to 25° C. So like Regel™, Mebiol® immediately and unavoidably would convert from liquid to solid upon injection into a 37° C. human body. Mebiol Inc. seems to be selling it as a culture medium and as a research-based (not FDA approved) occlusion means for tumors wherein the unavoidable solidification in the body chokes off blood flow and kills a tumor. The vendor has indicated that T_{gel} can be adjusted anywhere between 8° C. and 44° C. or so; however, there are no plans for a product above 37° C. The current product, MB-10, has the approximate T_{gel} of 20° to 25° C. An up-coming potential new product will likely have a T_{gel} of 32° C. or so.

Other companies have a variety of similar gels for drug-delivery in clinical trials or in production and these include, for example, TAP Pharmaceutical's Lupron Depot®, Alza Corp's Alzamer®, Atrix Labs Atrigel®, Southern Biosystem's Saber® Durect, Eurand's Biorise®, Durect Corp's Duros®, Genentech's Alkermes® and Schering-Plough's PEG-Intron™.

The above gels are all relatively recent materials for the many weeks- or months-long drug depot application and all solidify at or below body temperature with increasing temperature (as they are always employed in the prior art). Let us note specifically again that gellation-capable materials are sometimes used only in liquid form for their superb liquid-state dissolving or solvation power. In those cases (such as for the above-mentioned Hysolv® and Resolv® liquids), the material is specifically designed not to solidify during their prior art use as liquid-based super-solvents for drugs.

### BRIEF SUMMARY OF THE INVENTION

In accordance with an embodiment of the invention, a gel-like material is provided, the gel-like material having an on-demand state-change, whether the on-demand change is to an initial state, a final state or both, at least some of the gel present or delivered undergoing the change.

In accordance with another embodiment of the invention, a gel-like material is provided, the gel-like material having a spatially and/or temporally selective state-change, whether on-demand change is to an initial state, a final state or both, at least some of the gel present or delivered undergoing the change.

In accordance with yet another embodiment of the invention, a gel-like material is provided that selectively changes state due to gel contacting diseased tissue having a natural thermal or compositional contrast or artificially induced thermal or compositional contrast capable of causing state-change via a state-change parameter, independent of an on-demand nature or a spatial/temporal selective nature.

### BRIEF DESCRIPTION OF THE DRAWINGS

The sole FIGURE is a cross-sectional view of a portion of a human breast, showing cancerous tissue and treatment of the tissue in accordance with an embodiment of the invention.

### DETAILED DESCRIPTION OF THE INVENTION

### A. Definition of the Term “Gel”

In all cases herein by “gel” or “gel-like”, we more broadly mean any material which is intended to transition, at least once in one direction, from/to a liquid-like or flowable material to/from a semisolid, pseudo-solid or gel-like less flowable or non-flowable material.

Thus, this definition includes all prior art networked and other gels but can also include solutions of microgels, gel-emulsions, emulsions, colloidal gels, colloidal particulate gels, denaturing gels, thermoreversible gels, thermally responsive gels, non-gel materials having steep temperature vs. viscosity curves or ionic doping content vs. viscosity curves, or for that matter any networking material, Typically, but not exclusively, the inventive materials in their solid-like, gel-like or pseudo-solid states will be elastically, viscoelastically, viscoplastically or thixotropically deformable under mechanical loading. Most often they will be viscoelastic with small deformations and somewhat viscoplastic with further larger deformations. They may or may not incorporate other agents such as drugs, biological species or radiation sources, depending on our specific application. They will preferably be biocompatible for at least a useful period and may be biodegradable in some cases if we rely on the natural bodily processes to eventually clear the material from the body.

Some of our inventive gel-like materials will offer immediate on-demand clearing capability unlike all of the prior art. By “immediate” we mean as quickly as after a few seconds, minutes or hours such as after a medical or therapeutic procedure or treatment is delivered. Thus, our “gels” may be cold-set materials, warm-set materials or materials whose setting into the pseudo-solid state depends on manipulation of a non-thermal parameter such as solvent or water removal (or even addition) or manipulation of a dopant such as calcium, magnesium, potassium or sodium ions. Within the scope of “loading” or “doping” the gel with one or more beneficial agents, we include all manner of solid, liquid, gaseous or other agent materials including nanoparticles, microparticles, microfibers, microspheres, biological species and genetic species, whether or not they themselves contain or support additional or other agent materials beneficial to a patient. Also within scope of our invention are materials that undergo the flowability transition when exposed to light, radiation, particle beams, electron beams, or exposure to an energy field such as an electrical or magnetic field. In these cases, the physicist will appreciate that the parameter we are manipulating is exposure of the material to a beam or field having an intensity or polarization parameter or to a current caused thereby.

Herein, by gel or gel-like, we also mean a material that does contain or is capable of containing at least one gel or gel-like particle, if not being comprised of a bulk 100% gel or gel-like material across its full volume. Thus, a liquid suspension or emulsion of gel particles meets our definition as at least each such particle has gel-like behavior or potential.

### B. Introduction

We utilize gel-like materials, defined herein for our inventive purposes, which are changed from a flowable state to a non-flowable state and/or from a non-flowable state to a flowable state (in whole or in part) in a novel on-demand manner. Preferably, this is done while the gel-like material is already situated in or upon a patient's anatomy. The gel material may also undergo an additional state-change resulting from the act of depositing it in/on the body or resulting from the act of performing a surgery or therapy that exposes some gel to a varying state-change parameter. In any event, at least one such state-change takes place on-demand by manipulation or planned change of a physical parameter of the material or its immediate environment, such as of a temperature, pressure, ionic-content or solvent-concentration change. Prior art medical gels completely solidify immediately upon injection regardless of the practitioner's wishes as to where or when such solidification is to take place. Prior art medical gels also cannot reflow or reliquefy (or solidify) on-demand in whole or in part. Our inventive gels and gel methods all involve an on-demand state-change of at least some of the gel material present. By “on-demand” we mean at least initiated by a practitioner or his/her instructions at a time or times chosen or predetermined by the practitioner, the time(s) preferably being after the gel is introduced into or onto the body, usually seconds, minutes, hours or days after the gel introduction and any immediate, if any, state-change taking place upon said introduction.

A large class of materials known to at least one of (a) change flowability states or (b) be dissolvable after solidification includes both inorganic and polymeric gels and networks, many emulsions and microgels, as well as combined inorganic-polymeric gels and networks. We deliver or introduce the gel or gel-like material into or onto the patient's anatomy either as a flowable material or as an unflowable or much-less flowable gel-like material. This initial state may be assumed before deposition (for example in a syringe) or may be assumed after injection as by bodily heating from the patient upon said injected or otherwise delivered gel deposition. By “unflowable” we mean unflowable when in gellated or “firmed-up” form in the body and under the influence of bodily forces or surgeries/therapies that may need the gel to stay in place. We specifically note that with enough pressure or heating, for example, even an “unflowable” material meeting this definition can still be injected from a syringe or pressurized catheter in its unflowable form because we can provide very high injection pressure to force such flow-deposition. In any event, the gel-like material either remains in its deposited state, liquid or solid, or changes state fairly quickly as by body heating (or cooling) influences, thus providing the initial unflowable or flowable state. Typically, but not always, the initial state will be unflowable as discussed below.

What is different about our invention over the prior art is that some or all of the gel-like material at some point(s) in time, preferably while in/on the body, undergoes an on-demand or practitioner-initiated state-change from the initial state. By “practitioner-initiated” we mean the practitioner changes or directs to be changed a parameter at least some of the gel-like material or its surroundings that causes an on-demand state-change of the gel material. Thus, the practitioner could even direct a patient to later (or remotely) cause said parameter change and state-change if he/she does not himself/herself change the parameter. The on-demand change may take place seconds, minutes, hours, days, weeks, months or years after the initial state is assumed upon deposition. It may also or alternatively take place as the result of a delivered therapy or as a result of the use of a surgical implement. Typically, there will be at least one gel deposition, and in many cases one such deposition will define the beginning of a treatment cycle in a multideposition or multicycle treatment. We discuss below the doping of the gel with beneficial agents; however, some gels are known to themselves offer encouraging environments for cell growth or to have other therapeutic behavior and we include in the scope of our invention the use of gels which themselves, in undoped condition, provide a medical, surgery or therapy benefit anywhere in the body including remote from the gel site(s).

The deposited material, in its assumed or de facto unchanged initial state or in its on-demand changed state, whether flowable or unflowable, particularly if utilized as a carrier for a second therapeutic agent or dopant material, may serve one or more of several useful purposes including:
a) acting as a drug or beneficial-agent(s) delivery depot;
b) acting as a radiation or energy depot or source (the radiation source or energy being either doped into the gel material or being created by external influences applied to an in-situ gel);
c) acting to stop the flow of blood or of a bodily fluid as for bloodless surgery, (such as for liver or brain surgery);
d) acting as a sink for removal of bodily toxic contaminants such as heavy metals, radioactive substances or even biological or microbial species that react with or are captured by the gel;
e) acting to stop blood flow or to displace blood to reduce hemodynamic heat-transfer as for thermally enhancing a thermally-ablative tumor treatment or lesion-making process (may be a heating or cryo-cooling ablation procedure);
f) acting to starve a tumor or diseased/undesired tissues of any of nutrients, gases and ions as by reducing blood exposure (might also include a cancer drug(s));
g) acting as an attenuative dissipator (or mask) to enhance (or minimize) the heating/cooling performance of a thermal ablation procedure on specific tissues;
h) acting as a lumen or organ-cavity internal/external coating or filler for a variety of useful reasons, including drug delivery, stable organ shutdown, organ preservation or organ mechanical stability,
i) acting as an in-vivo cultivation site as for growing, cultivating or genetically-manipulating microbes or cells, such site possibly thereby acting as a “seed” for such cellular or tissue growth or as a depot to disperse such species in any manner into the body, wherein such grown, cultivated or processed species may involve genetic changes made possible in or by the gel;
j) if optically transparent at a useful wavelength, allowing for greater depth of penetration of therapeutic or diagnostic devices at that wavelength (e.g., photodynamic therapy, spectroscopy, fluorescence imaging); and/or
k) acting as an ongoing, long-lasting, emergency source of patient or human nourishment or maintenance as by depositing a gel including any of food, vitamins, enzymes, nutrients, proteins, minerals, etc. in a body portion such that the body can access it in a beneficial manner
l) acting as a contrast agent for an imaging modality such as MRI, PET, CATSCAN, Ultrasound, X-Ray, Fluoroscopy, Optical Imaging, Terahertz Imaging, Impedance Imaging

Many, but not all of our embodiments, have the as-deposited or quickly-assumed initial state as an unflowable state with the later on-demand state-change being a reflowing or dissolution of the initial unflowable material to become flowable and/or dissolvable material. This is because for many applications it is desired that the gel-like material be immobilized for a useful period by means of its own pseudo-solid viscosity, shear-strength or flow-resistance despite the normal functioning of the anatomy and any potential disruption caused by the delivery of the therapy or surgery itself.

### C. Our Invention

All of our inventive embodiments are fundamentally different than the prior art in at least one or both of two respects.
1) The first respect is that in many, but not all of our embodiments, we utilize a gel (per our definition herein unless otherwise indicated) that can, at least in part, be solidified (rendered usefully unflowable) and then reliquified/redissolved (rendered usefully flowable) such as by warming and subsequent cooling (or vice-versa, depending on the gel material and application). The solidification period may be from seconds to weeks or months, depending on the application. Existing gels delivered into the body exclusively utilize materials that solidify and then naturally biodegrade over an extended uncontrolled period of months, they do not reliquefy as by on-demand manipulation of a state-change parameter such as a temperature or ionic concentration. Our solidified gels will, at least, be capable of reflow/dissolution on demand if that option is desired to be exercised in the procedure.
2) The second respect is that in many, but not all of our embodiments, we utilize a gel (per our definition hereinafter unless otherwise indicated) which is, at least in part, rendered at least one of flowable/dissolved or non-flowable at least once in a selective manner, the term “selective” in this context meaning one or both of spatially selectively or temporally selectively. Existing gels non-selectively completely gel upon injection due to body heat. They also non-selectively completely biodegrade on their own slow schedule. So in summary, our gels can either or both of (a) be solidified and/or reflowed/dissolved on-demand and/or (b) be selectively solidified and/or reflowed/dissolved, preferably but not necessarily on-demand (we will provide examples below).

Given these unique respects, we will now outline the preferred basic embodiments of the invention.

### D. Embodiments

1. Unflowable to Flowable (and/or Flowable to Unflowable) with Use of “On-Demand” Capability

A gel or gel-like material is introduced into or onto a patient's anatomy, some or all of it is rendered on-demand unflowable or it itself becomes unflowable (unflowable in the body environment is the desired initial state) during or after the delivery at one or more anatomy sites. After a therapy, surgery or beneficial procedure that the unflowable gel supports is executed, some or all of the unflowable gel is again rendered flowable or is reliquified/dissolved (the final state) via on-demand manipulation of a state-transition parameter or via biodegradation. The reliquified/dissolved/biodegraded gel may then be removed naturally or artificially from the body. The medical procedure, in some cases, may itself render the gel reflowable in some applications. “On-demand” capability is used at least once in this embodiment to attain one or both of an initial state or final state.

2. Unflowable (or Flowable) to Flowable (or Unflowable)—with Spatial or Time Selectivity

This embodiment is similar to Embodiment #1 except for these optional differences:
a) The desired initial state may be flowable or unflowable, the final state being the other state.
b) A state transition of at least some material may be done in a spatially or temporally selective manner. This selective transition may be a transition (if any) to the initial state and/or a transition to a final state.
c) At least some material may undergo an on-demand state-transition.

Either or both of “on-demand” or “selective transition” capability is/are used at least once in this embodiment.

### E. Further Considerations

It should be immediately apparent that Embodiment #1 allows, for example, for the benefits of state-transitioning gels to be utilized in their solid form for controlled durations after which they are reliquified/dissolved/biodegraded.

It should also be immediately apparent that Embodiment #2 allows, for example, for the optional benefits of i) spatial or temporal selectivity of what specific portion of deposited gel undergoes state-change and/or when, ii) use of a procedure-supporting gel in its liquid or flowable state, iii) on-demand state-change.

Before we proceed to the supporting Figure, we shall now describe some specific applications for these unique and novel capabilities. Each of these applications may be practiced in at least one if not both of the above described embodiments.

### EXAMPLE (A)

### Bloodless Surgery

An organ, or portion thereof, such as a liver or brain, is infused with gel which displaces blood as it flows in and then solidifies or becomes unflowable such that it remains in place, displacing blood (or other bodily fluid such as urine or CSF). While gel-infused, a bloodless or minimally bloody surgery is performed by cutting tissues whose lumens and/or blood-perfusability is substantially plugged by the solidified gel. When the surgery/therapy is completed, the gel is reliquified by the practitioner or surgeon and blood is again readmitted (or forcefully infused) to the organ or portion thereof. Depending on the length of time the gel is infused, one may beneficially add an agent to the gel that provides life-supporting gases or nutrients to maintain sufficient tissue metabolism. As an example, oxygen could be infused in the gel by itself or in an added agent-carrier such as a perfluorinated oxygenated liquid constituent. Such flows of gel and/or blood might be due to natural perfusion/cardiac-pumping or may be due to artificially induced pressure gradients being applied. Such gel-plugs may comprise any shape, including high-aspect ratio shapes such as barrier sheets. In this manner, the “plugs” may or may not be cut through but they all control bleeding and/or bodily fluid or surgical/therapy fluids transport. Because of the two embodiments, we may perform such solidification locally or globally in an organ or in a gel that is deposited therein, at one point in time or at several times at one or more points. The gel may also promote cauterization or blood coagulation during surgical incision making. The gel may also or instead be used to close a wound or puncture.

### EXAMPLE (B)

### Trauma Care

To stop bleeding such as life-threatening massive bleeding, whether external or internal bleeding is involved, a gel material may be introduced into the body. The gel may be placed in one or both of locations whereat blood, bodily fluids, or gases (such as air in the lungs) normally reside. The objective is to stem blood flow and side effects of blood being where it should not be, such as in the gut or lungs. When the bleeding is stopped, regardless of how it is stopped, the gel may be reliquified. Such a gel may also contain agents such as the life-giving agent of Example (a) or such as blood-clotting drugs, for example. Likewise, a penetrating wound could be filled with such a gel-like material. In that case, one may also include added agents to fight infection and/or reduce pain. Note that in this embodiment, the gel itself may act to plug or block bleeding as by physical blockage. Because of our two embodiments, we have a choice as to whether we solidify all gel infused or we only selectively solidify some gel that is infused. As for other embodiments by infusion, we really mean delivery in any physical manner-such as by syringe, catheter, port, inhalation, ingestion, submergence, physical stuffing of a wound, spraying, etc.

### EXAMPLE (C)

### Temporary Blockage or Reroute of Blood Or Bodily Fluid (Urine, Cerebrospinal Fluid, etc.)

Think of this, at least in one variation, as a non-invasive clamp or suture. Essentially, one could easily selectively block (or reduce) the flow of a bodily fluid (e.g., blood) for a short period, such as to allow for a surgery to take place downstream without massive bleeding. This is advantageous because clamping has known undesirable side effects, such as plaque liberation, and is at least minimally invasive if not invasive. Such temporary blocking may also be to maximize the exposure of a tissue to be treated to a drug or radioactive therapy by avoiding the dilution or uncontrolled exposure of the beneficial agent to non-target tissues by blood-flow transport, for example. Included in our inventive scope is the use of heating or cooling means used to maintain a solidified state, whether such means are invasive or non-invasive.

### EXAMPLE (D)

### As a Contrast Agent

By “contrast agent” we mean enhancing the image contrast of an imaging modality and/or enhancing the absorbtion of a treatment or therapeutic energy, beam or particle-flux. In ultrasound, for example, it is difficult to image next to gas pockets. The unflowable gel would displace the air with a material, thereby minimizing uncontrollable reflections and echogenicity. In the treatment energy scheme, the gel is designed to enhance the absorbtion (or creation in the gel) of a treating energy, field or beam delivered external from the gel deposit. The absorbtion mechanism directly or indirectly provides treatment or therapy. The reader will realize that with enhanced absorption, one may reduce treatment time instead or as well. In this application, the gel may temporarily replace blood, bodily fluid, or bodily gases. Another possible related use is for some solidified in-vivo gel to act as an elastography stiffness calibration material.

### EXAMPLE (E)

### As a Temporary Drug Or Cell/Tissue Depot

This is for use when drugs, medicaments or biological or genetic species are to be delivered for a short period and/or must be removable at will. This includes all manner of medicaments, including those that leak out or diffuse out from the gel naturally, as well as those that are urged out of the gel, as by ultrasound breaking microcapsules of drug or purposeful pH change. Included here is the growth of therapeutic cells or tissues in or on such a gel, in which case the gel would typically include life-supporting nourishment therefore. Once the gel is removed or biodegraded, one may leave behind a growth of such cells and tissues, such as stem-cells, attached or juxtaposed to remaining tissues. Note that in the case of the gel-supporting growth of cells or tissues that remain behind after gel-removal or biodegradation, one leaves something behind after gel reflow beyond simply a drug. Thus, the gel in this case acts like a temporary cellular or tissue nursery. Such a gel nursery could even be infused into diseased tissues such as brain tissues wherein the nursery is growing new neurons.

### EXAMPLE (F)

### As a Temporary Source of Radiation

This is for use when the source or agent material needs to be removed at will, perhaps because it is too intense to leave it inside the patient for weeks. Upon reflow or reliquification, it may pass out of the body naturally or it may be removed by artificial means such as by suction and/or flushing. In this manner, one could dope such a gel with intense radiation sources and could place the gel, because of its solidification behavior, in regions where it would not normally remain for a useful treatment period, such as in a diseased lumen. Such a gel, like many others of the invention, may be provided with life-giving gas sources or nourishment to minimize damage due to stopped or slowed bloodflow (if bloodflow is stopped or slowed).

### EXAMPLE (G)

### As a Means of Organ or Tissue Preservation

By “organ” or “tissue” we mean any part of a body or limb or a whole body, limb or organ. By “preservation” we mean allowing for later re-use, revival or transplant of the organ, tissue or limb or individual. In this scheme, the unflowable gel-like material may be cooled or frozen during storage or transport. It is thought that a gel infused with life-giving nourishment and/or gases will also help the organ hold its correct shape, minimize bleeding during transplantation, and allow for more fragile anatomy portions such as nerve and neuronal structures. It may also serve, particularly if it supports or cultivates drugs or biological species, a bridging of function from two patients with somewhat different biological, genetic or biomolecular makeups. As examples, the gel might contain anti-rejection drugs, life-giving nourishers or gases, or growing cells or tissues that can functionally join their new host.

### EXAMPLE (H)

### A Means to Alter or Stop, at least Temporarily, Cooling Blood Flow or Bodily-Fluid Flow

There are many tissue thermal ablation technologies being utilized to necrose or lesion undesired tissues such as cancerous tissues. A common challenge to all of these is that bloodflow carries away much of the heat that is intended to necrose the tissue. By placing or forming our solidified gel at locations of normal high bloodflow (or bodily fluid flow), we can temporarily, for the sake of an easier or better-controlled ablation, stop or reduce this parasitic and often hugely-variable heat sinking. The same approach is applicable to cryosurgery ablation wherein warming blood is to be kept away from the treatment site.

### EXAMPLE (I)

### A Means to Provide Extra Rigidity or Filling/Smoothing

This could be used, for example, when a perforating or penetrating instrument is to be delivered into tissues. For example, such a gel would hold a lumen open for easier insertion of a syringe, catheter or port. Another example would be for lung surgery wherein the lung could be held in an inflated mode during medical intervention, therapy or surgery. Another is to fill out or firm up wrinkles or depressions in tissues for cosmetic reasons.

### Example (J)

### As a Mask for Harmful Radiation

As an example, a solidified gel could intercept treatment radiation which would otherwise be deposited in tissues that are not to be treated or are to be treated to a lesser degree. The same is true of directed photodynamic therapy wherein some tissue is to be protected. In this manner, the gel provides areal selectivity of the exposure beyond what the treatment radiation or beam can provide. Note that a gel could also be used as a “contrast agent” in the sense that it enhances absorption of the treatment radiation or light at desired sites.

### EXAMPLE (K)

### As a source of Nutrition

It might be desirable, for example, to infuse the stomach or digestive tract of starving persons (as by a disaster or catastrophe) with a gel-nourishment material. The advantage is avoiding the need for multiple or even one meal a day for a short period, thus furthering the benefit a finite number of rescuers can do. The gel might have as a manipulated parameter drinking water (a gel solvent). Intake of drinking water would control nutritional release. This follows the inventive scheme as the manipulated parameter is solvent manipulation and it is done with a practitioner's or clinician's guidance. In other words, the gel is not simply naturally biodegrading independent of the user's actions. The gel may also contain drugs, antibiotics, vitamins, etc. Such a gel might not be thermo-reversible but only solvent-reversible. Such a gel may also store or modulate bodily water. Such a gel may be arranged to be biodegradable such that it contollably releases a nourishing agent.

### EXAMPLE (L)

### As a Cancer Treatment or Surgery

This example is directed to the killing of cancerous or diseased tissues, such as by blood, oxygen and nutrient strangulation, drug treatment or radioactive exposure. The strangulation effect on tumor nourishment may or may not be enhanced or complimented by an agent delivered from the gel into the tissue.

We should note here that the gels of our invention may be incrementally solidified and/or reflowed or may be bulk-solidified or reflowed. An incremental reflow, for example, could be Example (k) above wherein water (solvent) intake incrementally reflows or dissolves the gel and delivers its useful nourishment or drug materials. These may also or instead diffuse or leach out of the solidified gel. Such a gel might also be bulk solidified (locally selectively or globally) and then incrementally reflowed or dissolved, for example.

### EXAMPLE (M)

### As a Means to Physically Stabilize or Position an Organ

In some surgeries and imaging modalities, organ perfusive or breathing motions either ruin or degrade the images or interrupt or disallow the desired surgical procedure. Filling the organ with the inventive gel would greatly stabilize deformable organs such as the heart, lungs, liver, brain, intestines, muscles, etc. Another use would be to space apart organs or tissues when one tissue is to be treated and the other is not to be treated. Such treatment could include, for example, radiation or ablation.

### EXAMPLE (N)

### To Displace Space for Food

In obesity-control, it is desired to reduce the volume of the stomach and other food storage/processing members. Our inventive gel, particularly in its unflowable state, could act to displace food and/or to cause the patient to feel full so as to stop eating. Because the gel may be arranged to be incrementally solidified (or dissolved/flowed), one could adjust the gel volume to an optimum volume and position.

One preferred embodiment of our inventive gel is a thermoreversible gel that solidifies with warming and reliquifies with cooling and is deliverable in the flowable liquid-like or flowable state. In some cases, the gel may be delivered to at least one treatment region of the patient's body whereat it is solidified. Only the gel in the treatment region is solidified while other gel, if any, outside that region is not solidified and remains liquid and may, in some cases, pass out of the body or be intentionally removed in liquid form. The warming may be that due to bodily heat, a radiation treatment, or directed invasive or non-invasive warming as by using an ultrasound beam.

Another embodiment of our inventive gel is one that is solidified and then controllably dissolved by a solvent or ionic solution for example.

Yet another embodiment of our inventive gel is one that is cooled to solidify and then rewarmed to reliquefy. In this case, the cooling may be to a body temperature or may be that due to the use of an invasive or non-invasive cooling means.

Depending on the particular application and gel formulation, one may provide external (or invasive/semi-invasive) heating or cooling to perform any of solidification or reflow or may rely on body temperatures for such solidification or reflow. Frequently, one of these transitions will be driven by a body temperature and the other by an invasive or non-invasive heater or cooling implement, including remote heaters or coolers when possible. One embodiment of a remote heater of the invention is directed heating via ultrasound exposure as applied non-invasively, semi-invasively or invasively. Ultrasound heating can be controllably and spatially delivered as the beamed ultrasound attenuates as heat in tissues. A second embodiment is cooling provided by a cooling probe, whether invasive or non-invasive.

One might also consider raising or lowering the temperature of an entire body or limb for one or the other or both of thermally-induced solidification or reflow.

For ultrasound heating of gel, diagnostic-like ultrasound consoles may be utilized, with few if any modifications at impressively low powers to both image the tissues and solidify the gel. Ultrasound color-doppler means or ultrasound-elastography may also be utilized to detect the solidification and/or reflow processes such that the modest warming, say 10° C. or so, does not have to be measured directly. Most existing ultrasound imaging consoles could warm tissues as much as 20° C., given proper software or microcode and regulatory allowance. Typically, our desired warming (or cooling) range is in the range of 3° to 15° C. above (or below) the local bodily ambient, depending on exposure time so as to minimize necrosis, unless, of course, necrosis is desired, in which case, a delta of 20° C. or more might be applied. Typically, such warming ultrasound would be directed as a beam such that it can be spatially localized in three dimensions. Typically, such cooling would be applied as by cryoprobes (cool/cold but not necessarily freezing) or whole-body or limb cooling.

Note that we may also utilize directed cooling as by the use of a cooled-tip (not necessarily as cold as cryogenic temperatures) penetrating lumen or syringe sunk into a breast tumor. The amount of heat that needs to be removed to render a gel nonflowable is very small compared to that needed to freeze tissues as in cryotherapy. Because of this, the cooling means can be smaller and less invasive. Included in the scope of our invention is the use of a cooling implement that may offer only modest cooling for gel phase-changing (solidification or reflow) or which may also offer cryotherapy with or without the gel being present. The gel may also be used as a freeze-mask in cases wherein cryotherapy is to freeze a tissue portion adjacent a portion that is not to freeze. In this case, the gel acts as a freeze-mask or an antifreeze.

Similar to our cooled probes, we may also utilize heated probes of an invasive, semi-invasive or minimally invasive nature. These may utilize any form of heating including resistive heating, RF heating, microwave heating, ultrasound heating, photodynamic or light-induced heating, for example. In the case of RF, light and microwave, for example, the heating may be caused remote from the probe(s) itself and may further be beamed or directed. Heating and/or cooling may also be applied as by use of a flowing coolant or heating medium-including flow of such through natural bodily passages and/or lumens, natural or otherwise.

The invention opens up numerous possibilities for minimally invasive or non-invasive surgery and therapy. We include in the scope of our invention the use of the invention in combination with first and/or second image-guidance or diagnostic equipment including, but not limited to, MRI, CATSCAN, ultrasound, optical or IR scopes such as endoscopes, fluoroscopy, X-ray, PET, thermography, infrared fluorescence, and OCT (optical coherence tomography). In a preferred embodiment, such imagery datasets are co-registered to each other and to the patient's anatomy portion being treated. In some applications, the imaging will be realtime and offer procedure guidance. In others, it will be a pregathered database that is co-registered with the patient's body and made available for moving around or in that body. One may beneficially use the invention together with a stereotactic or robotic implement manipulation means. We specifically note for the reader that, given precise imaging data and good anatomy coregistration therewith, one could utilize the invention without any realtime imaging, such as on a brain tumor. Our inventive gel can prevent undesired shifting of tissues as well. However, in some embodiments, we typically utilize ultrasound, as it can both offer gel-heating (for solidification or reflow depending on gel) as well as elastography-indication of gel solidification or reflow. Note that in principle, we can deliver the ultrasound heating even if the ultrasound transducer is not an imaging transducer. Note also that in addition to elastography, we can also utilize color Doppler flow-imaging, which would depict a lumen or perfusive tissue that is blocked by solidified gel as having no flow or reduced flow. It is also known by ultrasound practitioners that a gellated gel (vs. its liquid-like counterpart) is typically of somewhat higher attenuation and echogenicity and therefore appears somewhat different in an ultrasound image. One may, for example, purposely design a gel to gellate (solidify) with significant entrapped nucleated air bubbles. Such bubbles or microbubbels can serve as a means to see the solidified gel, for example.

By medicaments and drugs delivered by the gel (or grown in-situ by the gel) we wish to include the very latest anticancer-targeted drug strategies. To begin, some examples of present-day cancer-specific drugs include but are not limited to: Herceptin® (trastuzumab), Gleevec® (imatinib mesylate), Avastin® (bevacizumab), Iresa® (getfitinib) and Tarceva® (erlotinib HCL). In the future, however, we expect to see drugs and drug cocktails that target more than one cancer-driver or mechanism contributing to tumor growth, proliferation or metastasis, thus perhaps doing a better job at preventing the development of drug-resistance. Two known examples of such intended targeted drug strategies include those of Exelixis®. The first strategy includes a group of compounds known as spectrum-selective kinase inhibitors. The second strategy is to develop drugs which inhibit individual kinases that are points of convergence in critical signaling pathways employed by growth factor receptors to transmit their abberant signals in tumour cells.

An excellent way to indirectly monitor in-vivo gel temperature is to monitor whether it is gelled or liquefied as by ultrasonic imaging or elastography. In many of our embodiments, it is desirable to know when the flowability state-change has taken place (and where) but there is no need, per se, to know temperature, particularly if one is not carrying out a thermal therapy or surgery. Again, ultrasound imaging or elastography serves this purpose as can other imaging and elestography techniques.

At the moment, the only reliable and accurate means of direct non-invasive temperature measurement (in 2-D and/or 3-D spatial coordinates over time) during ablation procedures is a special and widely-known thermometry-mode of MRI or magnetic resonance imaging. This single choice may not change for years to come. Thus, we see large “heavy metal” medical companies such as GE and Siemens utilizing their MRI hardware in combination with ultrasonic or RF thermal ablator devices such as those mentioned above. It is indeed a good way to sell more MRI machines. Unfortunately, MRI equipment is very, very expensive, having price tags, if the necessary facilities and insurance are included, of 1 to 3 million dollars or more. Despite this, it appears that, with time, regulatory agencies may require such temperature mapping measures for ablation-monitoring, whereas such monitoring is not a current requirement but is practiced in many cases. This move would assure the practice of thermal ablation is expensive and limited in availability, as it would be locked into MRI availability and procedure costs. That would still be good for the patient, in terms of a better curative procedure becoming available; however, the cost implications are unhelpful for a medical industry already seen as having out-of-control costs.

What would fundamentally alter the playing field and accelerate the growth of cancer treatment would be a new “ablation” approach to treating cancer which does not require, necessarily, the use of MRI temperature monitoring, but still has the reasonably good efficacy of existing thermal ablation procedures (e.g., 75 to 90%, depending on ablator type, patient and other factors). We offer such a technique herein. Note that using imaging and elastography we can not only see the expected state transitions between flowable and unflowable, we can also see if the gel gets overheated as by nucleating bubbles or boiling. We therefore expressly include in the scope of our invention the use of the gel-like material in any of its forms to provide either or both of transition-information as well as temperature-control information.

The gel-like material may be administered in one or more of several manners such as the following:
a) Via a natural lumen as by a catheter: In this case, one may typically deliver the liquid-form (or at least flowable form when under injection/administration pressure) gel via a catheter into an artery or vein, for example. Depending on where that entry point is, the gel may be delivered body-wide systemically or may be mainly delivered to a particular limb, appendage or organ.
b) Via a needle or penetrating port: Examples of this approach include hypodermic injection of the gel, typically in liquid-form, or delivery through a syringe or cannula.
c) Delivery into or adjacent to an open wound as by either (a) or (b) or as by pouring into or immersion or spraying of the wound.
d) Ingestion, Submergence, Spraying, Dipping, Pouring, Inhalation, Eating.

It should be apparent that the state-change parameter we manipulate may be temperature or an ionic concentration, for example. This may be done directly, as by injecting the disposed gel with heat or an ionic liquid, or indirectly, as by depositing the gel in or past a tissue region that has been so conditioned, perhaps selectively. Thus, what matters is that the gel “experiences” the parameter change in whole or in part. We also bring this up for a special case. This special case is that wherein tumors known to be hotter than their surrounding tissues themselves cause a thermal state-change of deposited or flowing past gel material. Note here that a spatial selectivity is delivered by the anatomy itself because only the hotter tumor causes local solidification.

The present inventors again note that directed modest-power ultrasound beams may be capable of imaging and activating the gel and tissues of interest. Because the temperatures are quite modest, say 40° to 45° C. (a few degrees above body temperature) or so for solidification, there is less concern for overheating or over treating. Further, because we can monitor blood flow with color-doppler ultrasonic or laser techniques, we know whether blood flow is stopped or not, thereby offering assured under-treatment avoidance. The inventors expect that blood flow or perfusion will be completely stopped or substantially throttled for at least a minimum time at least once. Depending on the procedural medical purpose, one may include drugs, medicaments or nutrients in the solidified gel. These could include anything from anticancer drugs, antiinflammation drugs, molecular biology, vasodilators, pain-reducing drugs, narcotics, stem cells or even oxygen-providing species. Note again that thermal state-changes are only one type of state-change parameter we may utilize, such as solvent exposure, ionic concentration changes, etc.

The invention is not limited to ultrasound thermal activation or to ultrasound imaging. Clearly, any directed energy capable of delivering a heating effect in a target tissue can be used to activate the gel at one or more simultaneous points or regions, preferably in a non-invasive or minimally-invasive (e.g., intracavity) manner. Such heating ultrasound can also be utilized invasively and/or under blood or bodily fluids. Further, our invention may also be utilized with MRI or other “heavy-metal” imaging tools as, even then, it still avoids the risks of high-temperature heating if not also the need to do any MRI-temperature monitoring. One might still choose to monitor our low temperatures using MRI temperature mapping, but that would probably then be as much or more for targeting purposes than for temperature control.

Other clinical benefits are expected to be gained using the inventive embodiments disclosed herein. One of these is that thermal ablation techniques frequently cause localized boiling and/or cavitation in the focal regions of the beams(s). This behavior leads to a propagation of the lesion-front different than if the bubbling were not present and also contributes to defocusing or focal degradation in ultrasonic beams. The bubbling also causes the heating process to be nonlinear in nature-making it hard to predict and model accurately. An infused gel could suppress such bubbling if not also enhance ablative heat production.

Given that our heating is very modest and that we can achieve it with little or no bubbling, we have a virtually completely linear process that is predictable and capable of being modeled. A conventional ultrasound imaging transducer operating, for example, in CW-Doppler mode already heats tissues/bone on the order of 5° C. max. Therefore, it can be appreciated that with modest changes in “diagnostic” power-levels and microcode, one may cause our solidification heating over ranges of, say, 5° to 20° C. incremental temperature increase. One might also cool the patient or his/her target organ in order to minimize the peak temperature reached.

We include within the scope of our invention the inclusion of temperature-sensitive dyes or contrast agents, such as those mixed into the gel, for example, which allow the optical, infrared or acoustic monitoring of at least peak temperature. Note that we really only need the peak temperature, if that, as we can judge or throttle the additional delivery of heating power using the Doppler-detected blood flow changes or elastography. In fact, we can rely entirely or primarily on the Doppler to tell us we have reached the needed temperature. We can also place echogenic contrast agents in the gel that simply serve to tell us where it is and, roughly, how concentrated it is. This is a much easier challenge than producing a 3-D temperature map registered to anatomy.

An intended application of this invention is the treatment of breast cancer. In that process, we note that sonographers, acoustic clinicians, and surgeons have experience with needle-guided biopsy sampling wherein the imaging transducer also supports the needle manipulation. In our inventive application herein, we note that the gel delivery needle may be smaller in diameter than conventional biopsy sampling, and thereby less painful. The gel delivery needle may also carry a thermistor or other temperature sensor used to monitor the taught state-change temperature control. Such a needle may also provide the needed heating, cooling or ionic agents to switch the gel.

The present inventors also include as embodiments agents mixed into our inventive gels, including nanoparticles, such as recently demonstrated gold nanoparticles. These nanoparticles produce localized heat when irradiated or illuminated by the appropriate light or electromagnetic waves. Thus, they can act to selectively enhance ablation or therapeutic heating, for example. Nanoparticles are also known to allow for spectroscopic interrogation of tissues wherein they are resident or attached to reveal information about temperature and/or chemical bonding to targeted and/or decorated cells. We include in the scope of our invention the premixing of such particles into our gel or the post-mixing after such gels are already in place in-vivo. Thus, a deposited gel could beneficially act to “take-up” beneficial agents delivered on a wider systemic basis.

The present inventors believe that the cost of medicine can be reduced using the invention because procedures expected to require an MRI or other “heavy-metal” imaging apparatus may, in many cases, be carried out using only ultrasound diagnostic and biopsy-like equipment. We will now discuss our sole FIGURE in order to better detail a breast-cancer application of the invention.

The sole FIGURE depicts an inventive gel-treatment system integrated with an ultra-sound imaging system. The ultrasound system preferably images the targeted tissues and the relevant blood flow (using Doppler techniques, for example) and also delivers the desired targeted modest gel-solidification heating. Although not shown, the transducer and needle may be arranged to be part of a stereotactic system wherein the transducer and needle are held if not moved by a controlled reference frame, having, in some embodiments, a degree of coregistration with images taken with another imaging modality such as an MRI, PET, CATSCAN, or mammography image.

In the single FIGURE (not to scale), we see a human breast 1 containing a tumorous or malignant mass 2a and attendant and attached tumor vasculature 2b. The outside breast skin-surface is indicated as item 3, and the breast nipple as item 4. An ultrasound transducer 5 is depicted coupled to breast 1. In this particular example, the ultrasound transducer 5 conveniently serves two functions:
a) In some embodiments, it provides 3-D images of the tumorous region 2a, including incoming and/or outgoing vasculature and/or arterial paths 2b. Thus, the transducer 5 preferably has both B-Mode (black and white) imaging capability as well as color-doppler blood flow measurement capability. It may also allow elastography imaging to detect gel solidification/flow.
b) It provides solidification-warming or heating of our inventive gel in a designated region, such as in the tumor and/or its connecting vasculature.

The transducer 5, although not shown in this manner, may be held in a stereotactic work holder such that its spatial position and orientation with respect to the patient's breast is fixed (or controllably varied), such that other modality images may be coregistered to the ultrasound imaging space, and such that the breast might be held or otherwise positioned to avoid movement errors.

Transducer 5 is depicted as including an insertable needle 6 mounted on a transducer needle-guide 6a. This needle will be used to inject or deliver our thermoreversible gel and any drug, chemical, microbiological, genetic or nuclear constituents it may contain. It might also be utilized to do a realtime in-situ biopsy or to extract a biopsy sample for clinical study. Different needles 6 may be places in the needle holder 6a for these purposes. Ultrasound transducer needle-guides of both the transducer-mounted type 6a (shown) and stereotactic type (not shown) are widely known and are currently used for biopsy sampling purposes. Needle 6 is shown as having a lumen 6b and a distal orifice 6c as well as a connected reservoir and injection means 7 (in phantom) for the gel to be injected. The gel in the reservoir/injector, item 7, may be stored in the liquid or solid state but preferably in the liquid state such that it flows at modest pressure and is ready to be transferred without heating. In some embodiments, gel-flow is done using positive displacement but we include in the scope of our invention also the use of pressure-controlled, capillary-wicking or gravity delivery.

Transducer 5 is shown performing 3-D ultrasound imaging of the tumor region 2a and needle 6 is depicted as already inserted into the tumor 2a and/or tumor vasculature 2b in its upper-half portion 2e. Liquid gel 2c flows out of the needle lumen 6b/c into the tumor/vasculature region 2e. Note that for our example here the lower half of the tumor region 2f is not shown as currently (or yet) having gel filling it. The 3-D (or 2-D) volumetric imaging is being performed in the region generally defined by phantom lines 5d, 5d and 5e as would be expected, for example for a phased array ultrasound transducer 5. Typical transducers 5, such as the one shown, have a case 5a, a strain-relief 5b and a connecting cable 5c.

It will be noted in the FIGURE that the patient's breast 1 is shown in air 8, meaning it is not shown immersed as in a coupling water-bath. The invention's scope includes all known and future methods of coupling or directing imaging or ablative energies at or into the tissues or delivering drugs, medicaments, nuclear agents or microbiological agents in our gel or with our gel. This would include a medium being a liquid such as water and/or breast 1 being completely or partially held/on in a clamping mechanism.

We have previously noted that we have at least two choices: 1) preheat the target tissues and inject liquid gel for immediate solidification, and b) inject the gel and post-heat the target tissues and/or gel to cause the desired solidification at a selected site(s). For extended solidification periods, one may either deliver a low level of constant heating (as by acoustic attenuation heating) or a large initial preheating or occasional pulsed heating, both of which will remain warm for finite periods. Note that by using ultrasound imaging, we have several ways of monitoring solidification such as Doppler-flow and elastography or as by B-mode or other imaging of an echogenic solidified gel. Conversely, our gel may automatically solidify upon injection due to body heat or body chemistry but be on-demand removable as by our manipulation of a state-change parameter causing a state-change from an initial state to a final state.

Ultrasound practitioners will be aware that historically it has been virtually impossible to design an ultrasound transducer to perform both excellent ultrasound imaging as well as HIFU (high-intensity focused ultrasound) heating for necrosis involving substantial heating ablation of 25° to 90° C. Our breakthrough here, in terms of directed ultrasound heating, is that we are imaging and heating, but because the heating is very modest (compared to necrosis ablations), it is within the realm of what a good imaging transducer could do with the appropriate beam forming and pulsing microcode and minor heat sinking modifications. For our purposes herein, we need heating levels (on the low end) on the order of what CW-Doppler modes of ultrasonic imaging devices already deliver to just a few times that, say from 5° C. to 20° C., for example. These higher heating levels are still very low compared to HIFU necrosis peak temperatures of up to 90° C.

A variation of the invention herein is where the clinician or user arranges for the unflowable-form of the gel material to have higher acoustic-attenuation than the liquid form. This can be accomplished with high solidified gel polymeric-content. This measure allows a couple of things:
1) It means that the solidified gel needs less acoustic power than a less attenuative solidified gel in order to keep it solid or in order to attenuatively heat it for thermal ablation or necrosis procedures; and
2) It means that, if desirable, the gel can serve to heat the gel/tumor to a temperature higher than that obtainable with a lower attenuating solidified gel. This would add moderate-temperature necrosing capability without using a high-power HIFU transducer. Note that in approach (2) above, we may have at least two if not three effects acting to kill cancerous cells: 1) the stoppage of blood flow and starvation of metabolism, 2) the heating necrosis effect and, possibly, 3) the action of a drug or agent in or emanating from the gel. These may be simultaneous or sequential.

Our inventive apparatus and method may be utilized together or in concert with another imaging device 9 such as an MRI, CATSCAN, fluoroscopy, PETSCAN or static X-rays, for example. Such supportive images may be taken any of before, during or after at least a portion of the gel surgery or therapy is delivered. Any imaging means 9 (indicated as Imaging System #2) may have a line-of-sight such as those depicted by phantom lines 9a. We emphasize that imaging system 9 may be utilized one or more of before, during, or after the surgery/therapy. In this manner, the images from imaging apparatus 9 may be coregistered or overlaid with those of transducer 5. Alternatively, imaging system 9 might provide the only images utilized in the procedures.

We have frequently used the term “surgical” to describe our invention's purpose, but we emphasize that in many of its embodiments, it may be more therapeutic than surgical. Therapeutic examples include using the invention for drug delivery or in-situ radiation treatment for example, especially when the tissues do not need to be cut.

Delivery of our gel, preferably in liquid form, will typically be by at least one of a) needle, b) port, c) cannula, d) catheter, e) immersion, f) pouring, or g) spraying. Part of the delivery path or final resting place may include lumens, vasculature or other natural or inflated bodily cavities. The present inventors further anticipate the use of bandages and other wound-closure implements that have a source of (or can be coupled to) liquid gel that can be solidified in-situ in or under the bandage or implement. In the case of serious battlefield wounds, one could literally dump liquid gel upon wounds and let the body heat (or a supplemental heater) solidify it to stop bleeding. Then, at a later time, we manipulate our transition parameter to remove it. Alternatively, we manipulate the parameter to solidify it without body heat. Such use could take place using a compress.

One example of where it might be useful to incorporate oxygenation, nutritional or other electrophysiological constituents in a gel would be wherein the blood, either locally or systemically, is partially or fully replaced with our liquid gel. In this manner, the clinician could take his/her time addressing various treatment sites needing localized heating or surgery and would not have to worry about necrosis due to oxygen, nutrient or ionic starvation/imbalance. The present inventors specifically include in the scope of the invention the use of recently known perfluorocarbon (for example) blood-substitutes, particularly as part of the gel system itself. Oxygen is directly lethal to strict anaerobic organisms because of their inability to detoxify oxygen radicals. Oxygen-enhanced environments are therefore, bactericidal for anerobes like clostridia, the organisms responsible for fulminant infections like myonecrosis (gas gangrene) and tetanus. Indirect benefits of oxygen administration would include improved killing by neutrophils and macrophages, which generate and release reactive oxygen species during phagocytosis. Anaerobic infections are most commonly associated with operations involving the bowel or a hollow viscus (e.g., appendectomy, cholecystectomy, colectomy, gastrectomy, bile duct exploration, etc.), but may also occur in the respiratory tract, head and neck, female genital tract, and soft tissue.

This method of treatment might be used in place of or in conjunction with hyperbaric oxygen. In addition to oxygen, the gel could also be impregnated with antibiotics active against anaerobes such as clindamycin, metronidazole, penicillin, 3^{rd} and 4^{th} generation cephalosporins, and carbapenems. Moreover, the activity of some antibiotics is potentiated under conditions of increased oxygen tension. These antibiotics include aminoglycosides, vancomycin and sulfonamides. On the other hand, enhancing the gel with a gas like CO₂ may result in improved treatment of infections caused by obligate aerobes like Mycobacterium, for example.

Other possible adjuncts to the gel might include matrix metalloproteinases (MMPs). MMPs are identified and subdivided on the basis of their substrate specificity into collagenases, gelatinases, stromelysin, and membrane-type MMPs. An abcess is a localized collection of pus that is surrounded and walled off by fibrous tissue. The only way to effectively treat an abcess is to lance it, provided the abcess can be accessed. The action of MMPs has the ability to degrade the fibrous components of the abcess, thereby effectively lancing it, without surgery.

In addition, the gel might contain an anesthetic or other analgesics (morphine, aspirin, acetaminophen, etc.), which can be locally released or activated in-situ in order to reduce the pain of inflammatory or traumatic conditions like infections, osteoarthritis, rheumatoid arthritis, etc.

Other possible components of the gel could include:
1) thrombolytic agents for cerebral vascular accidents, myocardial infarctions, peripheral vascular disease;
2) chemotherapeutic agents such as methotrexate for the non-surgical treatment of ectopic pregnancies; and/or
3) calcium, phosphate, bisphosphanates for osteoporotic bone.
   F. Examples of Specific Gels Useful for the Applications Described

The first example gel is the above-mentioned Regel™, whose composition and preparation are taught in our provided reference. By varying the A and B block proportions as specifically described therein, we may set the state-transition temperature as taught therein.

The second example gel is Mebiol™ gel from Mebiol Inc. in Japan. Their website is at www.mebiol.co.jp, and their thermoreversible gel can be custom designed to have a desired phase-change temperature in a manner similar to the above. The liquid form is referred to as the sol form. The liquid form is hydrophilic. In the gel form, this gel is hydrophobic. Unlike hydrogels, the Mebiol gel is highly lipophilic and can be loaded with time-release or out-diffusing drugs as described above.

There are a variety of known thermoreversible gel systems, several based on polymeric water-solutions of polyoxypropylene and polyoxyethylene. The gel transition temperature is dependant on the polymer content and any additional additives as taught by our references. One or more of these may be manipulated to change the transition temperature.

In our cancer-attacking application, for example, one may incorporate drugs or other chemical (or even nuclear) constituents that attack the cancer cells directly or indirectly. This chemical or drug effect may take place one or both of while the gel is in the liquid or gel states. Clearly, even after a thermoreversible gel is allowed to reflow and depart from a tumor region, it may have already delivered a drug, chemotherapy constituent or radiation source, which locally remains (or whose effect remains). The gel may contain a radiosensitizer that is delivered to the tumor and potentiates the effect of radiotherapy on the tumor through the generation of highly toxic free radicals. Alternatively, the gel may deliver oxygen to the tumor cells, which oxygen may be toxic for the tumor cells as a result of oxygen free radical generation.

The gel application could also utilize a light-emitting diode or laser to potentiate the effect of photodynamic therapy. After administration of a photosensitizer drug that ideally accumulates preferentially in the tumor cells, the light-emitting diode in the gel might be used to activate the photosensitizer. In its activated form, the photosensitizer reacts with oxygen to create damaging singlet oxygen.

The gel may also be used as a physical shield or radiation barrier by impregnating it with radiation-impenetrable materials to protect sensitive tissues during radiotherapy of a certain area. Alternatively, the shield may be made virtual by impregnating the gel with bioreductive materials such as glutathione. These bioreductive molecules can attenuate the harmful effects of radiation on surrounding tissues by scavaging free radicals.

The gel may also contain ferromagnetic particles that may induce local hyperthermia at the tumor through the creation of electromagnetic heat.

The gel may act as a vector for the delivery of all kinds of drugs including, but not limited to, chemotherapeutic agents, analgesics, gases or antibiotics in a prodrug form. The addition of heat may cause the drug, for example, antibiotic, analgesic or chemotherapeutic agent, to be released into the milieu of the tumor through the rupture of a linker molecule binding it to the gel. Alternatively, prodrugs may be injected into the body that only become activated by the application of heat that may be generated in the vicinity of the gel.

Two gels (or a gel species and a nongel species) may contain elements that separately have no effect but, when mixed together, have activity or potentiate each other's activities, thereby making the gel itself a kind of prodrug or progel.

The gel may have esthetic implications; for example, adding the gel to the lips or to the breast such that it plumps with the administration of local heat from kissing or physical manipulation of the breast, respectively. Such a gel per our invention's theme would be, at least in part, convertible from flowable to unflowable or unflowable to flowable on-demand.

In the most general use of the invention, the gel is solidified or rendered unflowable at the selective treatment site using directed modest heating. Other gel, if any, may be washed out of the body by the natural excretion and metabolism processes. We expressly include in the scope of the invention the gel being solidified in at least three possible manners, regardless of from where the solidification heat is derived (from the body or from artificial means).
1) Complete occlusion by gel-filling and solidification.
2) Partial occlusion (or interior coating) as by partial solidification on the warmer walls of a lumen or cavity, for example.
3) Creation of free-floating gel particles or clumps that are subsequently deposited, at least in part, by a physical or chemical filtering action of the circulatory system or organs

In some embodiments, a solidifying gel-like material is a material that undergoes network-style gellation to become a gel. However, within the scope of our invention is solidification or becoming a semi-solid or less-flowable emulsion by any means, such as by freezing, congealing, thickening or phase-change. Molecular networking or cross-linking is not a requirement. In some embodiments, the solidification process is reversible in that a change of temperature in the opposite direction (or to more extreme levels) results in reliquification or at least physical breakdown or dissolution. Again, temperature being the manipulated state-parameter is a preferred but not required approach.

The present inventors anticipate the use of needle-like devices that may one or both of deliver the gel and cause in any manner a solidification/liquification temperature-change. The needle-like device may also provide some biopsy information, as by retrieving a sample or as by in-situ spectral or optical-analysis means.

The present inventors anticipate that existing ultrasound diagnostic equipment may be useable or adjustable by their manufacturers such that they can deliver the modest acoustic powers necessary to cause the needed heating. Further, cooled or heated needles or probes that puncture and penetrate the skin may alternatively or also be used. The needles or probes may be equipped with heat-delivery or heat-removal (cooling) means in a manner similar to or identical to existing thermal-ablation probes using microwaves, RF, ultrasound HIFU, lasers or cryogenic (or less cold) cooling mechanisms or devices. The ultrasound equipment may, in some embodiments, also be used for image-guidance or patient diagnosis or follow-up. An ultrasound system that can operate in both 3-D and 2-D would be quite attractive. Further, ultrasonic or laser-doppler flow-measurement techniques may provide information as to the state of solidification or liquefaction of the gel or solidification material.

Ultrasound transducers utilized for such imaging, solidification or reliquification may be, for example, phased arrays, mechanical-sector scanning devices, single-element devices, mechanically-focused devices, lens-focused devices, devices applied to the patient's external skin, or devices applied to tissues while inside the body as by a scope or incision-entry. They may also comprise intravascular transducers that one or both of deliver gel, remove gel, and/or provide imaging or state-change support or activation. It is not a requirement of the invention that any ultrasound probe that is used both images and heats gel; it may only image, it may only heat gel, or it may not be used at all. By “image” we mean at least one scanline.

The means for delivering, removing, state-transitioning or imaging gels or for performing any related or supported surgery or therapy may any one or more of non-invasive, minimally invasive or invasive.

The present inventors also anticipate the availability of the inventive solidifying material for battlefield or paramedic use or even for self-use for traumatic injuries. We include in our inventive scope a source of such gel already coupled to flow into or onto the body that can be activated by the victim himself (if conscious) or by a compatriot, as by application of localized heat or cold. Such emergency application might include the provision of such needed heatflow in a manner similar to that used to heat military rations or MREs or “meals ready to eat” wherein a chemical reaction is triggered in an attached heating element by the user (or eater of the meal).

We also include in the scope of our invention the use of our inventive gel or solidification material in or on bandages or dressings such that it solidifies upon bodily contact or, alternatively, by selective heat flow caused by the user or his compatriot inward or outward.

The inventive gel material may be delivered in one or more boluses or administrations and may also be administered in a metered manner from an external or implanted reservoir. At at least one point in time, the gel material will satisfy our definition of a gel. Before use, gel or unmixed/uncombined constituents thereof, may be stored in liquid, semi-solid or solid forms and may require combination with water or other consumable constituent to be readied for use. Prestoring of gel as a dry powder and mixing with sterile water or saline for subsequent use is a preferred approach. Storage of a gel ready for use will likely involve storage in a sealed and/or temperature controlled container or UV resistant sterile container.

What is claimed is:

1. A gel-like material with on-demand state-change, whether the on-demand change is to an initial state, a final state or both, at least some of the gel present or delivered under-going the change.

2. The gel-like material of claim 1 wherein the gel-like material's initial state is an unflowable or less flowable state.

3. The gel-like material of claim 1 wherein the gel-like material's initial state is a flowable or more-flowable state.

4. The gel-like material of claim 1 wherein the initial state is less flowable than the final state.

5. The gel-like material of claim 1 wherein the initial state is more flowable than the final state.

6. The gel-like material of claim 1 wherein a state-change is influenced by any one or more of a temperature, ionic concentration, compositional, solvent concentration, pressure or influence of an imposed energy or energy- field change.

7. The gel-like material of claim 1 wherein the gel-like material, in at least one state, acts as any one or more of a drug depot, medicament depot, depot or cultivation site for biological, microbiological or genetically desirable species, a therapeutic radiation source, a therapeutic radiation mask or screen, a tissue or cell growth site, an energy-attenuation enhancer, a heating enhancer, or an imaging contrast agent of any type.

8. The gel-like material of claim 1 wherein the gel-like material, in at least one state, acts as any one or more of a means to reduce or eliminate organ or tissue motion for imaging or surgical reasons.

9. The gel-like material of claim 1 wherein the gel-like material, in at least one state, acts as any of a means to reduce or stop blood flow, reduce or stop the flow of a bodily fluid, or reduce or stop the flow or perfusion or passage of any type of fluid.

10. The gel-like material of claim 1 wherein the gel-like material, while in or upon a patient or treatment subject, has any parameter measured, monitored or controlled, including any one or more of a temperature, ionic concentration, chemical concentration, solvent concentration, state-change indicator, elastic or viscoelastic stiffness, flowability indicator or pressure.

11. The gel-like material of claim 1 wherein the gel-like material, while in or upon a patient, acts to starve, kill, necrose, poison, radiate or otherwise biologically destroy or cause to become practically disfunctional an undesired tissue or tissue constituent.

12. The gel-like material of claim 1 wherein a directed or otherwise selective heating, cooling or other energy delivery or removal is utilized invasively, minimally invasively or non-invasively to cause or support a gel state or flowability change.

13. The gel-like material of claim 1 wherein the gel-like material introduced into or onto a patient or treatment subject undergoes, at least in part, any of the following:
a) immediate change to the initial state as influenced, for example, by body temperature or composition, the temperature or composition being one of natural or artificially manipulated;
b) on-demand change to the initial state;
c) on-demand change to the final state;
d) on-demand change to the final state, which allows for gel removal from a treatment site or from the body;
e) one or more on-demand state-changes;
f) two or more on-demand state-changes at two or more times;
g) on-demand change to an unflowable or less-flowable state;
h) on-demand change to a flowable or more flowable state;
i) spatially selective state-change, either on-demand or not on-demand; or
j) temporally selective state-change, either on-demand or not on-demand.

14. The gel-like material of claim 1 wherein the gel-like material is delivered into or onto a patient's or treatment subject's body, organ or tissue in either of a flowable or non-flowable state.

15. The gel-like material of claim 1 wherein a supported therapy or surgery includes one or more of:
a) bloodless or minimally bloody surgery;
b) enhancement of any ablation process or selective protection therefrom;
c) attacking diseased tissues, cells or bodily fluids;
d) acting as a drug, medicament or radiation source or depot;
e) acting to physically stabilize an organ, tissue or lumen for any purpose;
f) acting to preserve, maintain or promote cellular life or viability in a tissue, organ, biological or genetic material or bodily fluid whether the tissue is patient attached or not;
g) acting as a source of nutrition, water, vitamins, minerals, enzymes, proteins, caloric intake or food;
h) acting to displace space for food in the stomach or connected appendages; or
i) acting to serve a temporary or permanent cosmetic purpose.

16. The gel-like material of claim 1 wherein ultrasound heating is utilized to effect a state-change, said state change being one or more of a) spatially selective, or b) temporally selective.

17. The gel-like material of claim l wherein ultrasound imaging along or within at least one scanline or subvolume is used to monitor one or more of a state, state-change, blood flow, bodily fluid flow, or elastic property of a gel or tissue.

18. The gel-like material of claim 1 wherein ultrasound is utilized to any of: promote drug or medicament delivery from a gel, promote the cultivation of a biological or genetic species in or adjacent a gel, promote the state-change of any portion of a gel, or promote a directed state-change of any portion of a gel.

19. The gel of claim 1 wherein at-least some gel undergoes a state or flowability cha ge as a result of spatially or temporally selective or nonselective heat exchange.

20. The gel of claim 19 wherein the heat exchange involves at least one of:
a) a modest warming or heating insufficient to cause significant thermal necrosis of tissue;
b) a modest warming or heating of between 1 and 20° C. for at least a short period;
c) a modest cooling insufficient to cause significant thermal necrosis of tissue;
d) a modest cooling of less than 40° C. for at least a short period;
e) a sustained modest warming or cooling, sustained for the time needed for the medical surgery or therapy to be performed;
f) any spatially selective or temporally selective heat exchange;
g) attenuative heat generation as-caused by an impinging energy such as ultrasound, light, particle beams, RF energy or microwaves; or
h) heat generated in a heat-generating contrast agent.

21. The gel-like material of claim 1 wherein gel is released from a treatment site on-demand after a surgical or therapy procedure is carried out.

22. The gel-like material of claim 1 wherein “on-demand” includes any one or more of:
a) at a time of a practitioner's choosing a state-change takes place;
b) at a time of a practitioner's choosing a state-change is initiated; or
c) at a time of a patient's or treatment subject's choosing a state-change takes place or is initiated.

23. The gel-like material of claim 1 wherein “spatially selective” includes any one or more of:
a) at at least one anatomical position in or on a patient's body;
b) at a position in or on the body whereat gel is delivered or otherwise made available;
c) at a position in or on the body whereat gel is available or can be made available to deliver a therapeutic or surgical benefit;
d) at a position in or on the body whereat gel is available or can be made available to undergo a beneficial state-change;
e) at a diseased anatomy or tissue portion; or
f) at two or more spatially separated or temporally different simultaneous or sequential sites.

24. The gel-like material of claim 1 wherein an image dataset is utilized regardless of whether that dataset is collected pre-procedure or during the procedure.

25. The gel-like material of claim 1 wherein stereotactic or other controlled positioning means are utilized to provide spatial navigation or spatial references in support of a therapy or procedure.

26. The gel-like material of claim 1 wherein the gel-like material is at least one of delivered or removed from the patient using a syringe, port, catheter or other artificial lumen or by using a natural lumen or cavity.

27. The gel-like material of claim 1 wherein the gel-like material biodegrades for reasons of either a) it is inherently biodegradable, or b) it is rendered biodegradable on-demand.

28. The gel-like material of claim 1 wherein a state-change takes place at least one of: a) spatially or temporally selectively, b) spatially or temporally non-selectively, c) via on-demand completion or initiation.

29. The gel-like material of claim 1 wherein any of: a) the gel-like material is thermoreversible, b) the gel-like material is state-reversible at least once, c) the gel-like material transitions to and from a state by the effect of the same transition parameter being changed, d) changes to the initial state and the final state are caused by manipulation or change of the same state-transition parameter, e) changes to the initial state and the final state are caused by manipulation or change two different state-transition parameters, or f) one or more state-changes in at least some gel-like material is completed or initiated on-demand.

30. The gel-like material of claim 1 wherein the gel material assumes two or more states including the initial state and the final state.

31. A gel-like material with a spatially and/or temporally selective state-change capability, whether on-demand change is to an initial state, a final state or both, at least some of the gel present or delivered undergoing or capable of undergoing the change.

32. The gel-like material of claim 31 wherein the gel-like material's initial state is an unflowable or less flowable state.

33. The gel-like material of claim 31 wherein the gel-like material's initial state is a flowable or more-flowable state.

34. The gel-like material of claim 31 wherein the initial state is less flowable than the final state.

35. The gel-like material of claim 31 wherein the initial state is more flowable than the final state.

36. The gel-like material of claim 31 wherein a state-change is influenced by any one or more of a temperature, ionic concentration, compositional, solvent concentration, pressure or imposed energy or energy-field change.

37. The gel-like material of claim 31 wherein the gel-like material, in at least one state, acts as any one or more of a drug depot, medicament depot, depot or cultivation site for biological, microbiological or genetically desirable species, a therapeutic radiation source, a therapeutic radiation mask or screen, a tissue or cell growth site, an energy-attenuation enhancer, a heating enhancer, or an imaging contrast agent of any type.

38. The gel-like material of claim 31 wherein the gel-like material, in at least one state, acts as any one or more of a means to (a) reduce or eliminate organ or tissue motion for imaging or surgical reasons, and/or (b) space apart organs or tissues such that one can be protected from an adjacent surgery or therapy happening to the other.

39. The gel-like material of claim 31 wherein the gel-like material, in at least one state, acts as any of a means to reduce or stop blood flow, reduce or stop the flow of a bodily fluid, or reduce or stop the flow or perfusion or passage of any type of fluid including any useful foreign fluid supporting the therapy or surgery.

40. The gel-like material of claim 31 wherein the gel-like material, while in or upon a patient or treatment subject, has any parameter measured, monitored or controlled, including any one or more of a temperature, ionic concentration, chemical concentration, solvent concentration, state-change indicator, elastic or viscoelastic stiffness, flowability indicator or pressure.

41. The gel-like material of claim 31 wherein the gel-like material, while in or upon a patient, acts to starve, kill, necrose, poison, radiate, toxify or otherwise biologically destroy or cause to become practically disfunctional an undesired tissue or tissue constituent or disease-related species.

42. The gel-like material of claim 31 wherein a directed heating, cooling or other energy delivery is utilized invasively, minimally invasively or non-invasively, said heating or cooling involving one or more of:
a) use of an invasive implement used at least to exchange thermal energy;
b) use of a non-invasive beam used at least to deposit thermal energy;
c) use of any invasive, minimally invasive or non-invasive flowed coolant or heating liquid or gas;
d) use of any type of heat-exchange pad, probe, catheter, needle or port; or
e) delivery of any type of heating or cooling means to or adjacent the site of the desired state change.

43. The gel-like material of claim 31 wherein the gel-like material introduced into or onto a patient or treatment subject undergoes any of the following:
a) immediate change to the initial state as influenced, for example, by body temperature or composition, the temperature or composition being one of natural or artificially manipulated;
b) on-demand change to the initial state;
c) on-demand change to the final state;
d) on-demand change to the final state, which allows for gel removal from a treatment site or from the body;
e) one or more on-demand state-changes;
f) two or more on-demand state-changes at two or more times;
g) on-demand change to an unflowable or less-flowable state;
h) on-demand change to a flowable or more flowable state;
i) spatially selective state-change, either on-demand or not on-demand; or
j) temporally selective state-change, either on-demand or not on-demand.

44. The gel-like material of claim 31 wherein the gel-like material is delivered into or onto a patient's or treatment subject's body, organ or tissue in either of a flowable or non-flowable state.

45. The gel-like material of claim 31 wherein a supported therapy or surgery includes one or more of:
a) bloodless or minimally bloody surgery;
b) enhancement of any ablation process or selective protection therefrom;
c) attacking diseased tissues, cells or bodily fluids;
d) acting as a drug, medicament or radiation source or depot;
e) acting to physically stabilize an organ, tissue or lumen for any purpose;
f) acting to preserve, maintain or promote life or viability in a tissue, organ, biological or genetic material or bodily fluid whether the tissue is patient attached or not;
g) acting as a source of nutrition, water, vitamins, minerals, enzymes, proteins, caloric intake or food;
h) acting to displace space for food in the stomach or appendages; or
i) acting to serve a temporary or permanent cosmetic purpose.

46. The gel-like material of claim 31 wherein ultrasound heating is utilized to effect a state-change.

47. The gel-like material of claim 31 wherein ultrasound imaging is used to monitor one or more of a state, state-change, blood flow, bodily fluid flow, elastic property of a gel or tissue.

48. The gel-like material of claim 31 wherein ultrasound is utilized to any of: promote drug or medicament delivery from a gel, promote the cultivation of a biological or genetic species in or adjacent a gel, promote the state-change of any portion of a gel, or promote a directed state-change of any portion of a gel.

49. The gel-like material of claim 31 wherein gel is released from a treatment site on-demand after a surgical or therapy procedure is carried out.

50. The gel-like material of claim 31 wherein “on-demand” includes any one or more of:
a) at a time of a practitioner's choosing a state-change takes place;
b) at a time of a practitioner's choosing a state-change is initiated; or
c) at a time of a patient's or treatment subject's choosing a state-change takes place or is initiated.

51. The gel-like material of claim 31 wherein “spatially selective” includes any one or more of:
a) at at least one anatomical position in or on a patient's body;
b) at a position in or on the body whereat gel is delivered or otherwise made available;
c) at a position in or on the body whereat gel is available or can be made available to deliver a therapeutic or surgical benefit;
d) at a position in or on the body whereat gel is available or can be made available to undergo a beneficial state-change;
e) at a diseased anatomy or tissue portion;
f) at two or more spatially separated or temporally different simultaneous or sequential sites; or
g) at two or more separate diseased sites.

52. The gel-like material of claim 31 wherein an image dataset is utilized in support of the surgery or therapy regardless of whether that dataset is collected pre-procedure or during the procedure.

53. The gel-like material of claim 31 wherein stereotactic or other controlled positioning means are utilized to provide navigation or spatial reference in support of the surgery or procedure.

54. The gel-like material of claim 31 wherein the gel-like material is at least one of delivered or removed from the patient using a syringe, needle, tube, port, catheter or other artificial lumen or by using a natural lumen, vasculature or cavity or by inhalation, drinking, eating or excreting.

55. The gel-like material of claim 31 wherein the gel-like material biodegrades for reasons of either a) it is inherently biodegradable, or b) it is rendered biodegradable on-demand.

56. The gel-like material of claim 31 wherein a state-change takes place at least one of: a) spatially or temporally selectively, b) spatially or temporally non-selectively, c) via on-demand completion or initiation.

57. The gel-like material of claim 31 wherein any of: a) the gel-like material is thermoreversible, b) the gel-like material is state-reversible at least once, c) the gel-like material transitions to and from a state by the effect of the same transition parameter being changed, d) changes to the initial state and the final state are caused by manipulation or change of the same state-transition parameter, e) changes to the initial state and the final state are caused by manipulation or change two different state-transition parameters, or f) one or more state-changes in at least some gel-like material is completed or initiated on-demand.

58. The gel-like material of claim 31 wherein the gel material assumes two or more states including the initial state and the final state.

59. A gel-like material that selectively changes state due to the gel contacting diseased tissue having a natural thermal, compositional or genetic contrast or having an artificially induced thermal, compositional or genetic contrast capable of causing state-change via a state-change parameter, independent of an on-demand nature or a spatial/temporal selective nature.

60. The gel-like material of claim 59 wherein the gel-like material has an initial state that is an unflowable or less flowable state.

61. The gel-like material of claim 59 wherein the gel-like material has an initial state that is a flowable or more-flowable state.

62. The gel-like material of claim 59 wherein the gel-like material has an initial state and a final state and the initial state is less flowable than the final state.

63. The gel-like material of claim 59 wherein the gel-like material has an initial state and a final state and the initial state is more flowable than the final state.

64. The gel-like material of claim 59 wherein the gel-like material may undergo a state-change that is influenced by any one or more of a temperature, ionic concentration, compositional, solvent concentration, pressure or imposed energy or energy- field change.

65. The gel-like material of claim 59 wherein the gel-like material, in at least one state, acts as any one or more of a drug depot, medicament depot, depot or cultivation site for biological, microbiological or genetically desirable species, a therapeutic radiation source, a therapeutic radiation mask or screen, a tissue or cell growth site, an energy-attenuation enhancer, a heating enhancer, or an imaging contrast agent of any type.

66. The gel-like material of claim 59 wherein the gel-like material, in at least one state, acts as any one or more of a means to reduce or eliminate organ or tissue motion for imaging or surgical reasons.

67. The gel-like material of claim 59 wherein the gel-like material, in at least one state, acts as any of a means to reduce or stop blood flow, reduce or stop the flow of a bodily fluid, or reduce or stop the flow or perfusion or passage of any type of fluid including a foreign fluid used in support of the surgery or therapy.

68. The gel-like material of claim 59 wherein the gel-like material, while in or upon a patient or treatment subject, has any parameter measured, monitored or controlled, including any one or more of a temperature, ionic concentration, chemical concentration, solvent concentration, state-change indicator, elastic or viscoelastic stiffness, flowability indicator, applied energy field or pressure.

69. The gel-like material of claim 59 wherein the gel-like material, while in or upon a patient, acts to starve, kill, necrose, poison, radiate, toxify or otherwise biologically destroy or cause to become practically disfunctional an undesired tissue or tissue constituent.

70. The gel-like material of claim 59 wherein a directed heating, cooling or other energy delivery is utilized invasively, minimally invasively or non-invasively, directed meaning beamed or selectively spatially applied with any type of implement or tool.

71. The gel-like material of claim 59 wherein the gel-like material introduced into or onto a patient or treatment subject undergoes any of the following:
a) immediate change to an initial state as influenced, for example, by body temperature or composition, the temperature or composition being one of natural or artificially manipulated;
b) on-demand change to the initial state;
c) on-demand change to a final state;
d) on-demand change to the final state, which allows for gel removal from a treatment site or from the body;
e) one or more on-demand state-changes;
f) two or more on-demand state-changes at two or more times;
g) on-demand change to an unflowable or less-flowable state;
h) on-demand change to a flowable or more flowable state;
i) spatially selective state-change, either on-demand or not on-demand; or
j) temporally selective state-change, either on-demand or not on-demand.

72. The gel-like material of claim 59 wherein the gel-like material is delivered into or onto a patient's or treatment subject's body, organ or tissue in either of a flowable or non-flowable state.

73. The gel-like material of claim 59 wherein a supported therapy or surgery includes one or more of:
a) bloodless or minimally bloody surgery;
b) enhancement of any ablation process or selective protection therefrom;
c) attacking diseased tissues, cells or bodily fluids;
d) acting as a drug, medicament or radiation source or depot;
e) acting to physically stabilize an organ, tissue or lumen for any purpose;
f) acting to preserve, maintain or promote life or viability in a tissue, organ, biological or genetic material or bodily fluid whether the tissue is patient attached or not;
g) acting as a source of nutrition, water, vitamins, minerals, enzymes, proteins, caloric intake or food;
h) acting to displace space for food in the stomach or appendages; or
i) acting to serve a temporary or permanent cosmetic purpose.

74. The gel-like material of claim 59 wherein ultrasound heating of any type is utilized to effect a state-change.

75. The gel-like material of claim 59 wherein ultrasound imaging is used to monitor one or more of a state, state-change, blood flow, bodily fluid flow, elastic property of a gel or tissue.

76. The gel-like material of claim 59 wherein ultrasound is utilized to any of: promote drug or medicament delivery from a gel, promote the cultivation of a biological or genetic species in or adjacent a gel, promote the state-change of any portion of a gel, or promote a directed state-change of any portion of a gel.

77. The gel-like material of claim 59 wherein gel is released from a treatment site on-demand after a surgical or therapy procedure is carried out.

78. The gel-like material of claim 59 wherein “on-demand” includes any one or more of:
a) at a time of a practitioner's choosing a state-change takes place;
b) at a time of a practitioner's choosing a state-change is initiated; or
c) at a time of a patient's or treatment subject's choosing a state-change takes place or is initiated.

79. The gel-like material of claim 59 wherein “spatially selective” includes any one or more of:
a) at at least one anatomical position in or on a patient's body;
b) at a position in or on the body whereat gel is delivered or otherwise made available;
c) at a position in or on the body whereat gel is available or can be made available to deliver a therapeutic or surgical benefit;
d) at a position in or on the body whereat gel is available or can be made available to undergo a beneficial state-change;
e) at a diseased anatomy or tissue portion; or
f) at two or more spatially separated or temporally different simultaneous or sequential sites.

80. The gel-like material of claim 59 wherein an image dataset is utilized in support of the surgery or therapy regardless of whether that dataset is collected pre-procedure or during the procedure.

81. The gel-like material of claim 59 wherein stereotactic or other controlled positioning means are utilized to provide navigation or spatial references in support of the surgery or therapy.

82. The gel-like material of claim 59 wherein the gel-like material is at least one of delivered or removed from the patient using a syringe, needle, port, catheter or other artificial lumen or by using a natural lumen or bodily cavity.

83. The gel-like material of claim 59 wherein the gel-like material biodegrades for reasons of either a) it is inherently biodegradable, or b) it is rendered biodegradable on-demand.

84. The gel-like material of claim 59 wherein a state-change takes place at least one of: a) spatially or temporally selectively, b) spatially or temporally non-selectively, c) via on-demand completion or initiation.

85. The gel-like material of claim 59 wherein any of: a) the gel-like material is thermoreversible, b) the gel-like material is state-reversible at least once, c) the gel-like material transitions to and from a state by the effect of the same transition parameter being changed, d) changes to the initial state and the final state are caused by manipulation or change of the same state-transition parameter, e) changes to the initial state and the final state are caused by manipulation or change two different state-transition parameters, or f) one or more state-changes in at least some gel-like material is completed or initiated on-demand.

86. The gel-like material of claim 59 wherein the gel material assumes two or more states including an initial state and a final state.

87. The gel-like material of claim 59 wherein the compositional contrast is induced by one or more of:
a) the diseased or undesired tissue naturally having a unique biological, genetic, optical or electromagnetic signature different than healthy tissue; or
b) the diseased or undesired tissue being artificially given a compositional, biological, genetic, optical or electromagnetic signature via use of a targeted drug or contrast agent.
